(19) United States
(12) Patent Application Publication
(10) Pub. No.: US 2007/0129342 A1
(43) Pub. Date: Jun. 7, 2007

(54) Compositions Containing Ansamycin

(75) Inventors: Robert K. Mansfield, Carlsbad, CA (US); Edgar H. Ulm, Encinitas, CA (US)

Correspondence Address: WILSON SONSINI GOODRICH & ROSATI, 650 PAGE MILL ROAD, PALO ALTO, CA, 94304-1050, US

(73) Assignee: CONFORMA THERAPEUTICS CORPORATION, San Diego, CA (US)

(21) Appl. No.: 11/565,550

(22) Filed: Nov. 30, 2006

Related U.S. Application Data
(60) Provisional application No. 60/742,093, filed on Dec. 1, 2005.

(51) Int. Cl. A61K 31/395

(52) U.S. Cl. 514/183

(57) ABSTRACT

Provided are pharmaceutical compositions containing an oil phase and an aqueous phase, the oil phase including an ansamycin and less than 2% w/w oleic acid, wherein the ansamycin is geldanamycin, 17-aminogeldanamycin, 17-allyalamino-17-demethoxy-geldanamycin, compound 563, or compound 237 having the structures below, or a salt of any one of the aforementioned ansamycins

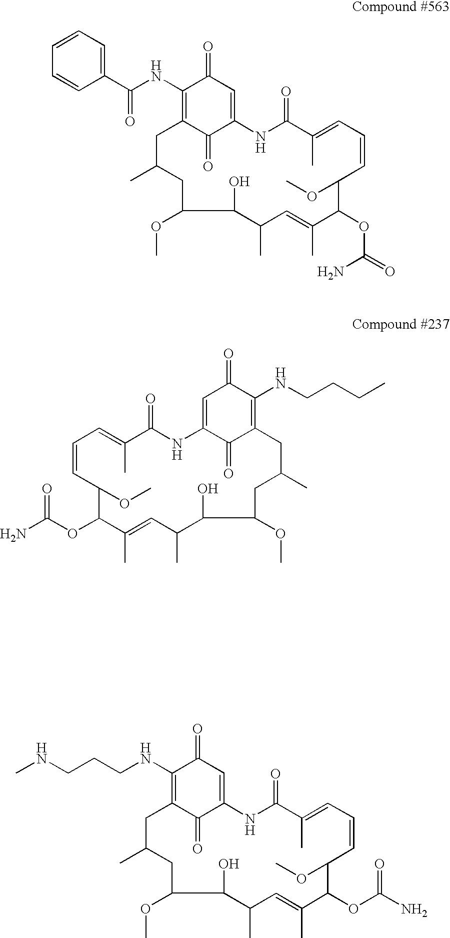

### RELATED APPLICATIONS

This application claims priority to U.S. Provisional Application Ser. No. 60/742,093, filed Dec. 1, 2005, which is herein incorporated by reference in its entirety (including all drawings). This application is also related to US Publications 2005/0176695, 20060014730, 2006/0067953, and 2006/0148776 and WO Publications 2003/026571, 2003/086381 and 2004/082676 all of which being incorporated by reference in its entirety.

### FIELD OF THE INVENTION

The invention relates in general to pharmaceutical compositions and methods of preparing and using the same. Specifically, the invention relates to compositions containing ansamycin (e.g., 17-allyalamino-17-demethoxy-geldanamycin (17-AAG)).

### BACKGROUND

17-allylamino-geldanamycin (17-AAG) is a synthetic analog of geldanamycin (GDM). Both molecules belong to a broad class of antibiotic molecules known as ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamyins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR (2001), 42, abstract 4474.

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J. TIBS 1999, 24, 136-141; Stepanova, L. et al. Genes Dev. 1996, 10, 1491-502; Dai, K. et al. J. Biol. Chem. 1996, 271, 22030-4). Studies further indicate that certain co-chaperones, e.g., HSP70, p60/Hop/Sti1, Hip, Bag1, HSP40/Hdj2/Hsj1, immunophilins, p23, and p50, may assist HSP90 in its function (see, e.g., Caplan, A. Trends in Cell Biol. 1999, 9, 262-68).

Ansamycin antibiotics, e.g., herbimycin A (HA), GDM, and 17-AAG are thought to exert their anticancerous effects by tight binding of the N-terminus ATP-binding pocket of HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., 1997, J. Biol. Chem., 272:23843-50). Further, ATP and ADP have both been shown to bind this pocket with low affinity and to have weak ATPase activity (Proromou, C. et al., 1997, Cell, 90: 65-75; Panaretou, B. et al., 1998, EMBO J, 17: 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. USA 96:1297-302; Schulte, T. W. et al., 1995, J. Biol. Chem. 270:24585-8; Whitesell, L., et al., 1994, Proc. Natl. Acad. Sci. USA 91:8324-8328). Ansamycins have also been demonstrated to inhibit the ATP-dependent release of chaperone-associated protein substrates (Schneider, C., L. et al., 1996, Proc. Natl. Acad. Sci. USA, 93:14536-41; Sepp-Lorenzino et al., 1995, J. Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and non-transformed cells alike and has been shown to be especially effective on a subset of signaling regulators, e.g., Raf (Schulte, T. W. et al., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Biol. Chem. 272:18694-18701; Smith, D. F. et al., 1995, Mol. Cell. Biol. 15:6804-12), v-src (Whitesell, L., et al., 1994, Proc. Natl. Acad. Sci. USA 91:8324-8328) and certain transmembrane tyrosine kinases (Sepp-Lorenzino, L. et al., 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812), CDK4, and mutant p53. Erlichman et al., Proc. AACR (2001), 42, abstract 4474. The ansamycin-induced loss of these proteins leads to the selective disruption of certain regulatory pathways and results in growth arrest at specific phases of the cell cycle (Muise-Heimericks, R. C. et al., 1998, J. Biol. Chem. 273:29864-72), and apoptsosis, and/or differentiation of cells so treated (Vasilevskaya, A. et al., 1999, Cancer Res., 59:3935-40).

In addition to anti-cancer and antitumorigenic activity, HSP90 inhibitors have also been implicated in a wide variety of other utilities, including use as anti-inflammation agents, anti-infectious disease agents, agents for treating autoimmunity, agents for treating stroke, ischemia, multiple sclerosis, cardiac disorders, central nervous system related disorders and agents useful in promoting nerve regeneration (See, e.g., Rosen et al. WO 02/09696 (PCT/US01/23640); Degranco et al. WO 99/51223 (PCT/US99/07242); Gold, U.S. Pat. No. 6,210,974 B1; DeFranco et al., U.S. Pat. No. 6,174,875. Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable with HSP90 inhibitors. Strehlow, WO 02/02123 (PCT/US01/20578). Still further HSP90 modulation, modulators and uses thereof are reported in Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/335,391, 60/128,593, 60/337,919, 60/340,762, 60/359,484 and 60/331,893.

Because of the poor water solubility properties of ansamycins, it is difficult at present to prepare ansamycin-containing pharmaceutical compositions, especially injectable intravenous formulations. To date, attempts have been made to use organic excipients (e.g., DMSO or castor oil derivative, Cremophor), lipid vesicles, and oil-in-water type emulsions, but these have thus far required complicated processing steps, harsh or clinically unacceptable solvents, and/or resulted in formulation instability. See generally Vemuri, S. and Rhodes, C. T., Preparation and characterization of liposomes as therapeutic delivery systems: a review, Pharmaceutica Acta Helvetiae 70, pp. 95-111 (1995); see also PCT/US99/30631, published Jun. 29, 2000 as WO 00/37050. DMSO, in addition to its hepatotoxic and cardiotoxic properties, is known to cause adverse events when administered to patients (nausea, vomiting, mal-odor), whereas cremophor is prone to induce hypersensitivity reactions and anaphylaxis in patients, who often require pretreatment with anti-histamines and steroids.

Commonly-owned US patent applications, 20060014730, 2006/0067953, and 2006/0148776, teach methods of preparing ansamycin compositions in the form of emulsions that do not require DMSO or cremophor to dissolve ansamycin. However, these emulsions have to be stored in frozen or lyophilized state for long term use, and thus causing inconvenience or difficulties during administration at the clinical sites (e.g., requires defrosting or rehydration and adjustment to a suitable concentration). There exists a need for ansamycin compositions that exhibit enhanced stability in refrigerated state or room temperature to increase the ease in handling the compositions during production and shipping and preparation for administration at the clinical sites.

### SUMMARY OF THE INVENTION

The present invention provides a pharmaceutical composition comprising an oil phase and an aqueous phase, the oil phase comprising an ansamycin and less than 2% w/w oleic acid, wherein the ansamycin is geldanamycin, 17-aminogeldanamycin, 17-allyalamino-17-demethoxy-geldanamycin, compound 563, or compound 237 having the structures below, or a salt of any one of the aforementioned ansamycins.

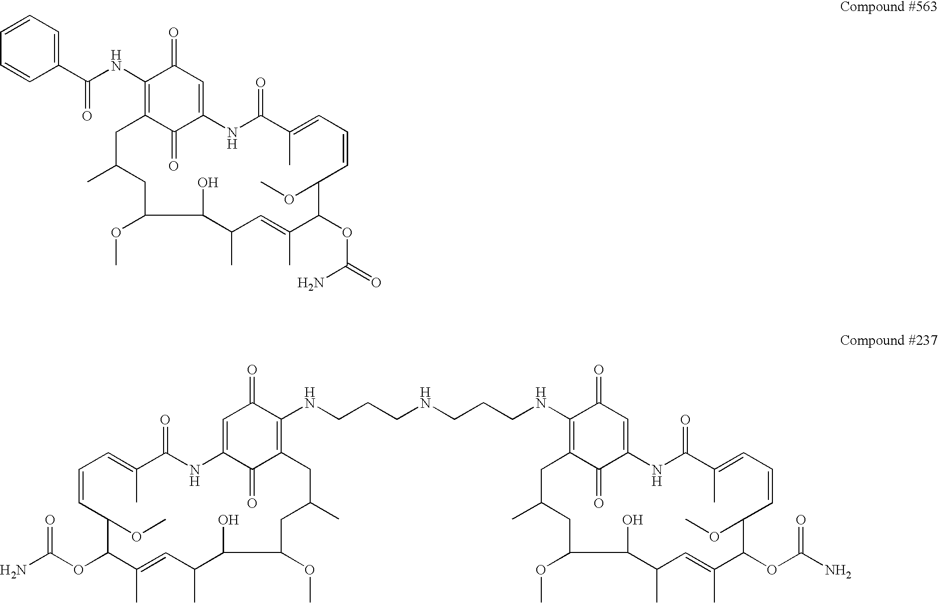

In one embodiment, the final concentration of the ansamycin ranges between about 0.5 to 4 mg/mL.

In another embodiment, the amount of oleic acid in the composition is no more than about 1% w/w of the pharmaceutical composition.

In yet another embodiment, the amount of oleic acid in the composition is between about 0.5% to 0.05% w/w of the pharmaceutical composition.

In a further embodiment, the pharmaceutical composition further comprises medium chain triglycerides. In still another embodiment, the amount of the medium chain triglycerides is no more than about 15% w/w of the pharmaceutical composition.

In still another embodiment, the pharmaceutical composition further comprises long chain triglycerides. In a further another embodiment, the amount of the long chain triglycerides is no more than about 7% w/w of the pharmaceutical composition.

In another embodiment, the pharmaceutical composition further comprises an emulsifying agent.

In a further embodiment, the invention provides a pharmaceutical composition of wherein the oil phase is about 5% to 30% w/w of the pharmaceutical composition.

In a further embodiment, the invention provides a composition wherein the final concentration of the ansamycin ranges between about 1 to 3 mg/mL; the amount of oleic acid in the composition is between about 0.5% to 0.05% w/w; the amount of the medium chain triglycerides ranges between about 7% to 13% w/w; the amount of the long chain triglycerides ranges between about 2% to 5% w/w; and the amount of lecithin ranges between about 5% to 8% w/w of the pharmaceutical composition.

Further embodiments of the invention, provide a composition wherein the mean droplet size is less than about 500 nm; the mean droplet size is less than about 150 nm; or the mean droplet size is about 80 nm.

In still another embodiment, the pH of the pharmaceutical composition ranges from about 5 to 8.

Yet another embodiment of the invention provides a pharmaceutical composition comprising an oil phase and an aqueous phase, the oil phase further comprising 17-allyalamino-17-demethoxy-geldanamycin and less than 2% w/w oleic acid, the pharmaceutical composition being stable at pH ranges from about 5 to 8 and temperature ranges between about 0° C. to 10° C. for at least 18 months.

Yet another embodiment provides a method of treating an individual having an HSP90 mediated disorder comprising administering to said individual an effective amount of a pharmaceutical composition according to the invention. The HSP90 mediated disorder may be one selected from the group consisting of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant diseases.

In yet another embodiment, the invention provides a method further comprising administering at least one therapeutic agent selected from the group consisting of cytotoxic agents, anti-angiogenesis agents and anti-neoplastic agents.

### BRIEF DESCRIPTION OF THE DRAWINGS

FIG. 1 shows the physical stability (mean droplet size) of six compositions that contained no oleic acid (C04H044, C05E011, C05F022, C05L043, C05L047, and C06A007) stored at frozen state (−20° C.).

FIG. 2 shows the physical stability (mean droplet size) of three compositions that contained 0.2% w/w oleic acid (N191-021, N191-058, and N191-150) at frozen state (−20° C.).

FIG. 3 shows the physical stability (mean droplet size) of compositions with and without oleic acid at room temperature. N191-021, N191-058, and N191-150 are three lots of composition with oleic acid whereas E05A002 does not contain oleic acid.

FIG. 4 shows the physical stability (mean droplet size) of six compositions that contained no oleic acid (C04H044, C05E011, C05F022, C05L043, and C05L047) at refrigerated temperature (5° C.).

FIG. 5 shows the physical stability (mean droplet size) of three compositions that contained 0.2% w/w oleic acid (N191-021, N191-058, and N191-150) at refrigerated temperature (5° C.).

### DETAILED DESCRIPTION OF THE INVENTION

The terms “evaporating” and “lyophilizing” do not necessarily imply 100% elimination of solvent and solution, and may entail lesser percentages of removal (e.g., about 95% or more).

The term “hydrating” or “rehydrating” means adding an aqueous solution, e.g., water or a physiologically compatible buffer such as Hanks's solution, Ringer's solution, or physiological saline buffer.

The term “about” is meant to embrace deviations of 20% from what is stated. The term “inclusive” when used in conjunction with the term “between” or “between about” means including the endpoints of the stated range.

As used herein, the term “stable” refers to the properties of a composition of the present invention. High stability at refrigerated temperatures (e.g., 0-10° C. or 2-8° C.) and room temperature (in comparison to similar compositions without oleic acid) is a characteristic of a composition of this invention. Typical, at room temperature and pH values of about 5-8 (e.g., 5.5-7), such an oleic acid-containing composition has a mean droplet size that increases no more than 100 nm (or even 50 nm) for at least 3 months; for refrigerated temperatures (e.g., 0-10° C. or 2-8° C.) and pH values of about 5-8 (e.g., 5.5-7), such an oleic acid-containing composition has a mean droplet size that increases no more than 50 nm (or even 35 nm) for at least 12 months. Further, if 17-AAG is present in a composition of the present invention, the major two degradation products of 17-AAG, RS-A and 17-AG, are found to be no more than about 2.5% (e.g., 1%) and 7.5% (e.g., 5%) w/w, respectively, in a 12-month period.

“Oils” include fatty acids and glycerides containing the same, e.g., mono-, di- and triglycerides as known in the art. The fatty acids and glycerides for use in the invention can be saturated and/or unsaturated, natural and/or synthetic, charged or neutral. “Synthetic” may be entirely synthetic or semisynthetic as those terms are known in the art. The oils may also be homogenous or heterogeneous in their constituents and/or origin.

The terms “short,” “medium” and “long,” when used to describe a carbon chain (e.g., in a fatty acid or triglyceride), refer to, respectively, less than 8 linear carbon atoms, 8 to 12 linear carbon atoms, and greater than 12 linear carbon atoms.

A “physiologically acceptable carrier” refers to a carrier or diluent that does not cause significant irritation to an organism and does not abrogate the biological activity and properties of the administered compound.

An “excipient” refers to a substance added to a pharmacological composition to further facilitate administration of a compound. Examples of excipients include but are not limited to calcium carbonate, calcium phosphate, various sugars and types of starch, cellulose and cellulose derivatives, gelatin, vegetable oils and polyethylene glycols. These can also be physiologically acceptable carriers, as described above, e.g., sucrose. Further falling within the definition of excipient are bulking agents. A “bulking agent” generally provides mechanical support for a formulation. Examples of such agents are sugars. Sugars as used herein include but are not limited to monosaccharides, disaccharides, oligosaccharides and polysaccharides. Specific examples include but are not limited to fructose, glucose, mannose, trehalose, sorbose, xylose, maltose, lactose, sucrose, dextrose, and dextran. Sugar also includes sugar alcohols, such as mannitol, sorbitol, inositol, dulcitol, xylitol and arabitol. Mixtures of sugars may also be used in accordance with this invention. Various bulking agents, e.g., glycerol, sugars, sugar alcohols, and mono and disaccharides may also serve the function of isotonizing agents, as described above. It is desirable that the bulking agents be chemically inert to drug(s) and have no or extremely limited detrimental side effects or toxicity under the conditions of use. In addition to bulking agent carriers, other carriers that may or may not serve the purpose of bulking agents include, e.g., adjuvants and diluents as well known and readily available in the art.

An “effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the route of administration, the condition being treated, and the individual being treated. Factors such as clearance rate, half-life and maximum tolerated dose (MTD) have yet to be determined but one of ordinary skill in the art can determine these using standard procedures.

Components of a Composition of the Present Invention

Ansamycin

The term “ansamycin” is a broad term which characterizes compounds having an “ansa” structure which comprises any one of benzoquinone, benzohydroquinone, naphthoquinone or naphthohydroquinone moieties bridged by a long chain. Compounds of the naphthoquinone or naphthohydroquinone class are exemplified by the clinically important agents rifampicin and rifamycin, respectively. Compounds of the benzoquinone class are exemplified by geldanamycin (including its synthetic derivatives 17-AAG and 17-N,N-dimethylamino-ethylamino-17-demethoxygeldanamycin (DMAG)), dihydrogeldanamycin and herbamycin. The benzohydroquinone class is exemplified by macbecin. Ansamycins and benzoquinone ansamycins according to this invention. Ansamycins and benzoquinone ansamycins according to the invention may be synthetic, naturally occurring, or a combination of the two, i.e., “semi-synthetic”, and may include dimers and conjugated variant and prodrug forms. Some exemplary benzoquinone ansamycins useful in the processes of the invention and their methods of preparation include but are not limited to those described, e.g., in U.S. Pat. No. 3,595,955 (describing the preparation of geldanamycin), U.S. Pat. Nos. 4,261,989, 5,387,584, and 5,932,566. Geldanamycin is also commercially available, e.g., from CN Biosciences, an Affiliate of Merck KGaA, Darmstadt, Germany, headquartered in San Diego, Calif., USA (cat. no. 345805). The biochemical purification of the geldanamycin derivative, 4,5-Dihydrogeldanamycin and its hydroquinone from cultures of Streptomyces hygroscopicus (ATCC 55256) are described in International Application Number PCT/US92/10189, assigned to Pfizer Inc., published as WO 93/14215 on Jul. 22, 1993, and listing Cullen et al. as inventors; an alternative method of synthesis for 4,5-Dihydrogeldanamycin by catalytic hydrogenation of geldanamycin is also known. See e.g., Progress in the Chemistry of Organic Natural Products, Chemistry of the Ansanzycin Antibiotics, 33:278 (1976). Other ansamycins that can be used in connection with various embodiments of the invention are described in the literature cited in the “Background” section above. In a composition of the present invention, the final concentration of the ansamycin (e.g., 17-AAG) is typically about 0.5-4 mg/mL (e.g., 1-3 mg/mL or 2 mg/mL).

Long Chain Triglycerides

“Long chain triglycerides” are triglyceride compositions having fatty acids greater than 12 linear carbon atoms in length. A common source of these is vegetable oil, e.g., soy oil or soy bean oil, which typically contains 55-60% linoleic acid (9,12-octadecadienoic acid), 22% oleic acid (cis-9-octadecenoic acid), and lesser amounts of palmitic and stearic acid. The amount of long chain triglycerides typically present in a composition of this invention is no more than about 7% w/w (e.g., about 2-5% w/w) based on the weight of the composition.

Medium Chain Triglycerides

“Medium chain triglycerides” as used herein are triglyceride compositions having fatty acids ranging in size from 8-12 linear carbon atoms in length, and more preferably 8-10 carbon atoms in length. Various embodiments of the invention include the use of Miglyol® 812N (Condea Vista Co., Cranford, N.J., USA). Miglyol® 812N contains roughly 50-65% caprylic acid (8 carbons) and 30-45% capric acid (10 carbons). Caproic acid (6 carbon atoms) is also present, up to a maximum of about 2%, as is Lauric Acid (12 carbons). Present in still a lesser amount (1% max) is Myristic acid (14 carbons). Other medium chain triglycerides that can be used in a composition of the present invention include Miglyol® 810, 818, 829, and 840, and other well-known medium chain triglycerides. Miglyol 812N has monographs in the European Pharmacopeia as medium chain triglycerides, the British Pharmacopeia as fractionated coconut oil, and the Japanese Pharmacopeia as caprylic/capric triglycerides. Other sources of medium chain triglycerides include coconut oil, palm kernel oil, and butter. The amount of medium chain triglycerides typically present in a composition of this invention is about 3-10% w/w (e.g., about 5-8% w/w) based on the weight of the composition.

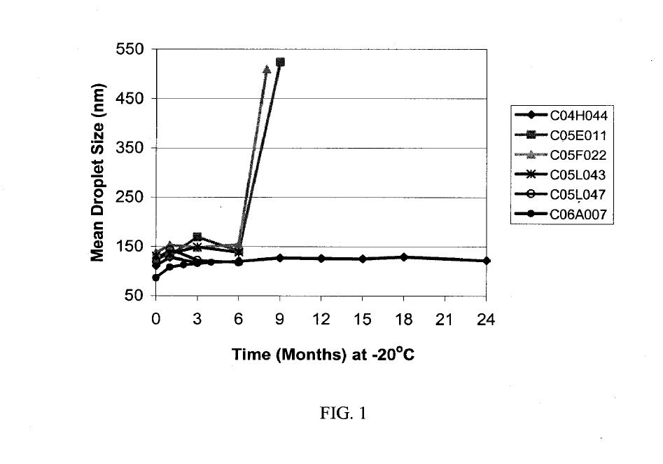

As described in commonly owned patent application, US 2006/0148776, Miglyol® 812N, when administered rapidly, can cause sedation due to the metabolic release of octanoate. During the intravenous infusion in rats of 17-AAG emulsion (Miglyol® 812N oil) sedation was observed at infusion rates greater than 1.1 gm total lipid/kg/hr. See FIG. 1 of related US application 2006/0148766. Sedation was also noted in dogs given intravenous infusions of the 17-AAG emulsion formulation at rates greater than 1.13 gm total lipid/kg/hr. To counter this, long chain triglyercides (e.g., soybean oil) were added as described above to compete with the metabolism of Miglyol 812N in-vivo to reduce octanoate fatty acid produced during intravenous infusions. In the soybean oil/Miglyol 812N CF237 emulsions, no sedation was observed acutely in rats at infusion rates of up to about 40 gm total lipid/kg/hr. Thus, the combination of soybean oil with Miglyol 812N greatly improves tolerability of the CF237 emulsion formulation with regard to sedation. Similarly, no sedation was observed in monkeys administered six doses of the CF237 emulsion formulation as an intravenous infusion of 12 mL formulation/kg/hr, and no vein irritation was observed.

Short Chain Triglycerides

“Short chain triglycerides” are triglyceride compositions having fatty acids less than 8 linear carbon atoms in length. This can be optionally present in a composition of the present invention.

Emulsifying Agents

“Emulsifying agents” are synonymous with “surfactants” and include but are not limited to phospholipids such as lecithins. “Lecithins” are naturally occurring mixtures of diglycerides of stearic, palmitic, and oleic acids, linked to the choline ester of phosphoric acid. The term surfactant or emulsifying agent also includes phosphatidylcholine, which distinct compound is well known. Examples of emulsifying agents for use with the invention are soy lecithin, e.g., Phospholipon 90G (PL9OG, American Lecithin Company, Oxford, Conn., USA) and soy phosphatidylcholine, e.g., Lipoid S-100 (Lipoid KG, Ludwigshafen, Germany). Phospholipon 90G has previously been used in parenteral nutritional products such as Intralipid® at a concentration of about 1.2%, Doxil® (doxorubicin) at about 1%, Ambisome® (amphotericin B) at about 2%, and Propofol® at about 1.2%. In the case of the latter, see, e.g., U.S. Pat. No. 6,140,374. The amount of surfactant/emulsifying agent typically present in a composition of this invention is about 3-10% w/w (e.g., about 5-8% w/w) based on the weight of the composition.

Examples of anionic surfactants include sodium lauryl sulfate, lauryl sulfate triethanolamine, sodium polyoxyethylene lauryl ether sulfate, sodium polyoxyethylene nonylphenyl ether sulfate, polyoxyethylene lauryl ether sulfate triethanolamine, sodium cocoylsarcosine, sodium N-cocoylmethyltaurine, sodium polyoxyethylene (coconut)alkyl ether sulfate, sodium diether hexylsulfosuccinate, sodium a-olefin sulfonate, sodium lauryl phosphate, sodium polyoxyethylene lauryl ether phosphate, perfluoroalkyl carboxylate salt (manufactured by Daikin Industries Ltd. under the trade name of UNIDINE DS-101 and 102).

Examples of cationic surfactants include dialkyl(C₁₂-C₂₂)dimethylammonium chloride, alkyl(coconut)dimethylbenzylammonium chloride, octadecylamine acetate salt, tetradecylamine acetate salt, tallow alkylpropylenediamine acetate salt, octadecyltrimethylammonium chloride, alkyl(tallow)trimethylammonium chloride, dodecyltrimethylammonium chloride, alkyl(coconut)trimethylammonium chloride, hexadecyltrimethylammonium chloride, biphenyltrimethylammonium chloride, alkyl(tallow)-imidazoline quaternary salt, tetradecylmethylbenzylammonium chloride, octadecyidimethylbenzylammonium chloride, dioleyidimethylammonium chloride, polyoxyethylene dodecylmonomethylammonium chloride, polyoxyethylene alkyl(C₁₂-C₂₂)benzylammonium chloride, polyoxyethylene laurylmonomethyl ammonium chloride, 1-hydroxyethyl-2-alkyl(tallow)-imidazoline quaternary salt, and a silicone cationic surfactant having a siloxane group as a hydrophobic group, a fluorine-containing cationic surfactant having a fluoroalkyl group as a hydrophobic group (manufactured by Daikin Industries Ltd. under the trade name of UNIDINE DS-202).

Examples of nonionic surfactants include polyoxyethylene lauryl ether, polyoxyethylene tridecyl ether, polyoxyethylene cetyl ether, polyoxyethylene polyoxypropylene cetyl ether, polyoxyethylene stearyl ether, polyoxyethylene oleyl ether, polyoxyethylene nonylphenyl ether, polyoxyethylene octylphenyl ether, polyoxyethylene monolaurate, polyoxyethylene monostearate, polyoxyethylene monooleate, sorbitan monolaurate, sorbitan monostearate, sorbitan monopalmitate, sorbitan monostearate, sorbitan monooleate, sorbitan sesquioleate, sorbitan trioleate, polyoxyethylene sorbitan monolaurate, polyoxyethylene sorbitan monopalmitate, polyoxyethylene sorbitan monostearate, polyoxyethylene sorbitan monooleate, polyoxyethylene polyoxypropylene block polymer, polyglycerin fatty acid ester, polyether-modified silicone oil (manufactured by Toray Dow Corning Silicone Co., Ltd. under the trade names of SH3746, SH3748, SH3749 and SH3771), perfluoroalkyl ethyleneoxide adduct (manufactured by Daikin Industries Ltd. under the trade names of UNIDINE DS-401 and DS-403), fluoroalkyl ethyleneoxide adduct (manufactured by Daikin Industries Ltd. under the trade name of UNIDINE DS-406), and perfluoroalkyl oligomer (manufactured by Daikin Industries Ltd. under the trade name of UNIDINE DS-451).

Oleic Acid

Oleic acid is an ionizable, mono-unsaturated omega-9 fatty acid with emulsification properties. It can be found in various animal and vegetable oils (e.g., olive oil). The amount of oleic acid present in a composition of the present invention is no more than 1% w/w (e.g., about 0.5-0.05% w/w or about 0.2% w/w). Since the dissociation constant of oleic acid is about 5, it is likely that the pH of the composition would have an impact on the effectiveness of oleic acid in stabilizing the droplet size.

It should be noted that other secondary emulsifiers (e.g., dimyristylphosphatidylglycerol (DMPG), Solutol HS15, and Tween 80) were tested at refrigerated temperature for droplet size stability improvement. It was found that Solutol HS15 and Tween 80 did not improve the droplet size stability and DMPG resulted in a viscous emulsion that would be difficult to draw a syringe while oleic acid showed improved stability without affecting other properties such as viscosity.

Sucrose

Sucrose is used as a bulking agent in the present invention. Sucrose is believed to allow for potential stability enhancement of the formulation by forming a dispersion of the oil droplets containing the active ingredient in a rigid glass. Polyvinylpyrrolidone (PVP) can be used to replace sucrose. The amount of bulking agent (e.g., sucrose) present in a composition of the present invention is no more than about 12% w/w (e.g., about 7-8% w/w).

Others

To prevent or minimize oxidative degradation or lipid peroxidation, antioxidants, e.g., alpha-tocopherol and butylated hydroxytoluene, and preservatives such as edentate may be included in addition to, or as an alternative to, oxygen deprivation (e.g., formulation in the presence of inert gases such as nitrogen and argon, and/or the use of light resistant containers).

Pharmaceutical acceptable co-solvents may also be added to the composition to further enhance the solubility of the ansamycins. Many suitable co-solvents that are known in the art may be used. Exemplary solvents includes, but are not limited to, glycerol, labrafil (apricot kemol Oil PEG-6 esters), labrasol (PEG-8 caprylic/capric glycerides), polyethylene glycol 400, Tween 80, Solutol HS15, propylene carbonate, Transcutol HP (ethoxydiglycol), and glycofurol.

Preparation of a Composition of the Present Invention

In general, the first step of a method of preparing a composition of the present invention is the dissolution of an ansamycin. As shown in Example 6 below, ethanol can be used to facilitate the dissolution of ansamycin into the oil phase of the composition. It is most common to first dissolve the ansamycin (e.g., 17-AAG) in the ethanol using sonication or heat followed by addition of oil phase components (e.g., long/medium chain triglyceride, oleic acid, and emulsifying agents) to the composition. Stirring and sonication are often necessary to effect mixing and dissolution of all the components. Ethanol is then removed by evaporation before the aqueous phase is added.

Alternatively, a composition of the present invention can be prepared by dissolving an ansamycin in the oil phase directly (without using ethanol) and mixing with aqueous phase. The two phases are separately prepared and then combined. The ratio of the two phases in a primary emulsion can be about 4:1 (aqueous phase: oil phase) (i.e., about 20% oil-in-water emulsion). It should be noted that ratios different from 4:1 can also be used. The primary emulsion is then microfluidized to reduce the droplet size (e.g., to about 80 nm mean droplet size), then sterile filtered and filled into the final container closure system under aseptic conditions. A general process flow for preparing a 17-AAG containing composition (in a 100 kg batch) is described below in Example 5.

Gentle heating could be used to facilitate the dissolution of ansamycin into the oil phase (e.g., about 40-60° C.). It should be noted that the elevated temperature should be adjusted based on the melting point of the ansamycin (which varies somewhat from one to another). For example, a lower melting point form of 17-AAG (prepared through crystallization of 17-AAG from isopropanol rather than ethanol) can even be dissolved into the oil phase at room temperature.

Note that 17-AAG degrades at higher rates when exposed to elevated temperatures for prolonged periods of time. Care (e.g., implementation of temperature control) should be taken when dissolving 17-AAG in heated oil phase.

A few buffer systems (citrate, phosphate, and L-histidine) were evaluated for use in a composition of the invention but such systems resulted in compositions with high viscosity and/or low stability. Thus, a composition of the present invention is used without being buffered. In unbuffered states, the pH gradually decreases at refrigerated temperatures and appears to stabilize at about pH 6. In preparing a composition of this invention, the pH of the emulsion is adjusted to about 7.5 (with, e.g., NaOH) prior to size reduction (since adjusting the pH of CNF1010 post size reduction leads to separation of the emulsion). The pH decreases during size reduction by 0.5-1.5 pH units.

The resulting composition is then emulsified, homogenized, or microfluidized (see description below) to achieve the desired mean droplet size. Sterilization is then employed to ensure that the composition is suitable for pharmaceutical use.

Emulsification and Microfluidization

Emulsions comprising an oil phase and an aqueous phase are widely known in the art as carriers of therapeutically active ingredients or as sources of parenteral nutrition. Emulsions can exist as either oil-in-water or water-in-oil forms. If, as is the case in the current instance, the therapeutic ingredient is particularly soluble in the oil phase the oil-in-water type is the preferred embodiment. Simple emulsions are thermodynamically unstable systems from which the oil and aqueous phases separate (coalescence of oil droplets). Incorporation of emulsifying agent(s) into the emulsion is critical to reduce the process of coalescence to insignificant levels.

Emulsification can be effected by a variety of well-known techniques, e.g., mechanical mixing, vortexing, and sonication. Sonication can be effected using a bath-type or probe-type instrument.

Microfluidizers are commercially available (e.g., Model 110S microfluidizer, Microfluidics Corp., Newton, Mass. and are further described in, e.g., U.S. Pat. No. 4,533,254) and make use of pressure-assisted passage across narrow orifices to reduce the size of the droplets in an emulsion. Pressure assisted extrusion through various commercially available polycarbonate membranes may also be employed. The composition of this invention may be microfluidized at high pressure (e.g., 16,000-19,000 psi) to reduce the particle size of the dispersion from about 5 μm to 0.1-0.5 μm or less (mean particle size).

Sterilization

Sterilization can be achieved by filtration, which can include a pre-filtration through a larger diameter filter, e.g., a 0.45 micron filter, and then through smaller filter, e.g., a 0.2 micron filter (e.g., a sterile 0.2 micron Sartorius Sartobran P capsule filter (500 cm²) at pressure up to 60 psi. The filter medium can be cellulose acetate (Sartorius-Sartobran™, Sartorius AG, Goettingen, Germany).

Characterization and Use of a Composition of the Present Invention

Characterization and Assessment of Chemical and Physical Stability

Phospholipids and degradation products may be determined after being extracted from emulsions. The lipid mixture can then be separated in a two-dimensional thin-layer chromatographic (TLC) system or in a high performance liquid chromatographic (HPLC) system. In TLC, spots corresponding to single constituents can be removed and assayed for phosphorus. Total phosphorous in a sample can be quantitatively determined, e.g., by a procedure using a spectrophotometer to measure the intensity of blue color developed at 825 nm against water. In HPLC, phosphatidylcholine (PC) and phosphotidylglycerol (PG) can be separated and quantified with accuracy and precision. Lipids can be detected in the region of 203-205 nm. Unsaturated fatty acids (e.g., oleic acid) exhibit high absorbance while the saturated fatty acids exhibit lower absorbance in the 200 nm wavelength region of the UV spectrum.

Emulsion visual appearance, mean droplet size, and size distribution can be important parameters to observe and maintain (determine physical stability). There are a number of methods to evaluate these parameters. For example, dynamic light scattering and electron microscopy are two techniques that can be used. See, e.g., Szoka and Papahadjopoulos, Annu. Rev. Biophys. Bioeng., 9:467-508 (1980). Morphological characterization, in particular, can be accomplished using freeze fracture electron microscopy. Less powerful light microscopes can also be used.

Emulsion droplet size distribution can be determined, e.g., using a particle size distribution analyzer such as the CAPA-500 made by Horiba Limited (Ann Arbor, Mich., USA), a Coulter counter (Beckman Coulter Inc., Brea, Calif., USA), or a Zetasizer (Malvern Instruments, Southborough, Mass., USA).

In addition, the chemical stability of the composition, in particular, the active ingredient, ansamycin, e.g, 17-AAG, can be assessed by HPLC after extraction of the composition/emulsion. Specific assay procedures can be developed that allow for the separation of the therapeutically active ansamycin from its degradation products. The extent of degradation can be assessed either from the decrease in signal in the HPLC peak associated with the therapeutically active ansamycins and/or by the increase in signal in the HPLC peak(s) associated with degradation products (e.g., 17-AG or RS-A in the case of 17-AAG). Ansamycins, relative to other components of the emulsion components, are easily and quite specifically detected at their absorbance maximum of 345 nm.

Modes of Formulation and Administration

Although intravenous administration is preferred in various aspects and embodiments of the invention, one of ordinary skill will appreciate that the methods can be modified or readily adapted to accommodate other administration modes, e.g., oral, parenteral, aerosol, subcutaneous, intramuscular, intraperitoneal, rectal, vaginal, intratumoral, or peritumoral.

Compositions of the invention, as described previously, are well suited for immediate or near-immediate parenteral administration by injection, e.g., by bolus injection or continuous infusion. In the latter method of administration, a continuous intravenous delivery device may be utilized to maintain a constant concentration in the patient. An example of such a device is the Deltec CADD-PLUS™ model 5400 intravenous pump. Compositions for injection may be presented in unit dosage form, e.g., in ampoules or in multi-dose containers, with an added preservative, e.g., edentate. As discussed previously, the compositions of the invention can be stored in an inert environment, e.g., in ampoules or other packaging that are light-resistant or oxygen-free.

Pharmaceutically acceptable compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Some excipients and their use in the preparation of formulations have already been described. Others are known in the art, e.g., as described in PCT/US99/30631, Remmington's Pharmaceutical Sciences, Meade Publishing Co., Easton, Pa. (most recent edition), and Goodman and Gilman's The Pharmaceutical Basis of Therapeutics, Pergamon Press, New York, N.Y. (most recent edition).

Dose Range

A phase I pharmacologic study of 17-AAG in adult patients with advanced solid tumors determined a maximum tolerated dose (MTD) of 40 mg/m² when administered daily by 1-hour infusion for 5 days every three weeks. Wilson et al., Am. Soc. Clin. Oncol., abstract, Phase I Pharmacologic Study of 17-(Allylamino)-17-Denzethoxygeldanamycin (AAG) in Adult Patients with Advanced Solid Tumors (2001). In this study, mean±SD values for terminal half-life, clearance and steady-state volume were determined to be 2.5±0.5 hours, 41.0±13.5 L/hour, and 86.6±34.6 L/m². Plasma Cₘₐₓ levels were determined to be 1860±660 nM and 3170±1310 nM at 40 and 56 mg/m². Using these values as guidance, it is anticipated that the range of useful patient dosages for formulations of the present invention will include between about 0.40 mg/m² and 225 mg/m² of active ingredient. Standard algorithms exist to convert mg/m² to mg drug/kg bodyweight.

Treatment of HSP90-mediated Diseases

In some method embodiments, the preferred therapeutic effect is the inhibition, to some extent, of the growth of cells characteristic of a proliferative disorder, e.g., breast cancer. A therapeutic effect will also normally, but need not, relieve to some extent one or more of the symptoms other than cell growth or size of cell mass. A therapeutic effect may include, for example, one or more of 1) a reduction in the number of cells; 2) a reduction in cell size; 3) inhibition (i.e., slowing to some extent, preferably stopping) of cell infiltration into peripheral organs, e.g., in the instance of cancer metastasis; 3) inhibition (i.e., slowing to some extent, preferably stopping) of tumor metastasis; 4) inhibition, to some extent, of cell growth; and/or 5) relieving to some extent one or more of the symptoms associated with the disorder.

In some embodiments, the compositions of the present invention are used for the treatment or prevention of diseases that are HSP90-dependent/mediated. In some embodiments, the compositions are used in the manufacture of a medicament. In other embodiments, the compositions are used in the manufacture of a medicament for the therapeutic and/or prophylactic treatment of diseases and conditions that are HSP90-dependent. Examples of such diseases and conditions include disorders such as inflammatory diseases, infections, autoimmune disorders, stroke, ischemia, cardiac disorder, neurological disorders, fibrogenetic disorders, proliferative disorders, tumors, leukemias, chronic lymphocytic leukemia, acquired immunodeficiency syndrome, neoplasms, cancers, carcinomas, metabolic diseases, and malignant disease. The fibrogenetic disorders include but are not limited to scleroderma, polymyositis, systemic lupus, rheumatoid arthritis, liver cirrhosis, keloid formation, interstitial nephritis and pulmonary fibrosis.

The compositions of the instant invention may also be used in conjunction with other well known therapeutic agents or methods that are selected for their particular usefulness against the condition that is being treated. For example, the instant compositions may be useful in combination with known anti-cancer and cytotoxic agents or other treatment methods (e.g., radiation). Further, the instant methods and compositions may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, including ribozymes and antisense targeted to VEGF receptors, angiostatin and endostatin.

Examples of antineoplastic agents that can be used in combination with the compositions and methods of the present invention include, in general, and as appropriate, alkylating agents, anti-metabolites, epidophyllotoxins, an antineoplastic enzyme, a topoisomerase inhibitor, procarbazine, mitoxantrone, platinum coordination complexes, biological response modifiers and growth inhibitors, hormonal/anti-hormonal therapeutic agents and haematopoietic growth factors. Exemplary classes of antineoplastic include the anthracyclines, vinca drugs, mitomycins, bleomycins, cytotoxic nucleosides, epothilones, discodermolide, pteridines, diynenes and podophyllotoxins. Particularly useful members of those classes include, e.g., canninomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

Advantages of Compositions of the Present Invention

Ansamycin-containing compositions containing no oleic acid (e.g., those described in the working examples of US 2006/0014730 and 2006/0148776) have to be stored frozen (at about −20° C.) or lyophilized to preserve the physical stability of the product. Even at frozen state, stability could vary between lots of ansamycin-containing compositions without oleic acid. Based on stability data, one lot (C04H044) was stable for two years at −20° C. and other lots (e.g., lot C05E011 and C05F022) were stable for only 6 months. See FIG. 1. All six compositions shown in FIG. 1 are identical in composition (see Table 1 below) and contain no oleic acid. These compositions were prepared using methods similar to that described in Example 5.

TABLE 1
Composition of the compositions shown in FIG. 1.

| Ingredient | Composition (% w/w) |
|---|---|
| 17-allyalamino-17-demethoxy-geldanamycin (17- | 0.2 |
| AAG) | |
| Miglyol 812, NF (Medium Chain Triglycerides) | 9.9 |
| Soybean Oil, USP (Long Chain Triglycerides) | 3.3 |
| Phospholipon 90G (Soy lecithin) | 6.6 |
| Oleic Acid, NF | 0.0 |
| Sucrose, NF | 7.5 |
| EDTA, USP | 0.005 |
| Sodium Hydroxide, NF | To adjust pH |
| Water for Injection, USP | q.s. (sufficient |
| | quantity) |

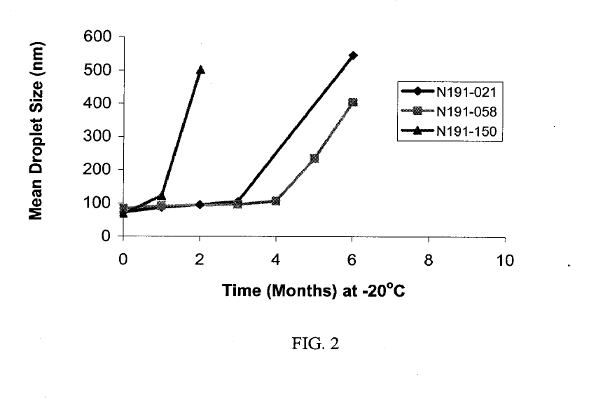

On the other hand, the droplet size stability for CNF1010 containing oleic acid is not stable when stored at −20° C. (see FIG. 2) with similar lot-to-lot variability observed with compositions that do not contain oleic acid (see FIG. 1). The three lots of oleic acid-containing compositions all contain the same composition as that described in Table 2 below and they were prepared using methods described in Example 5.

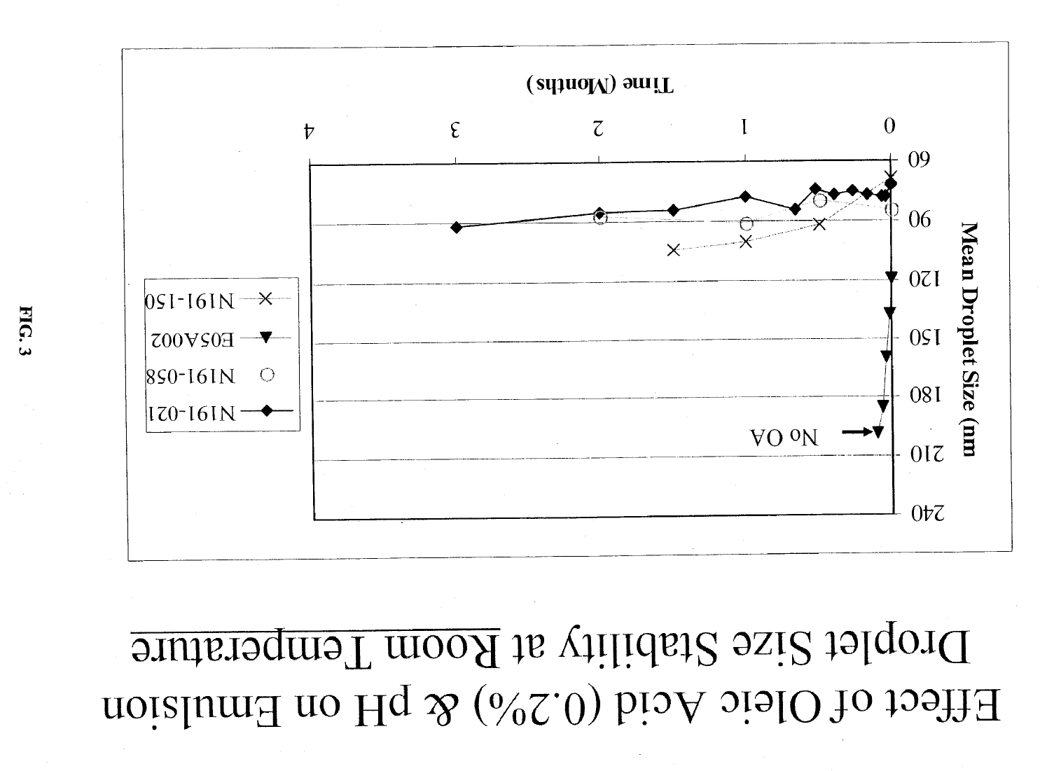

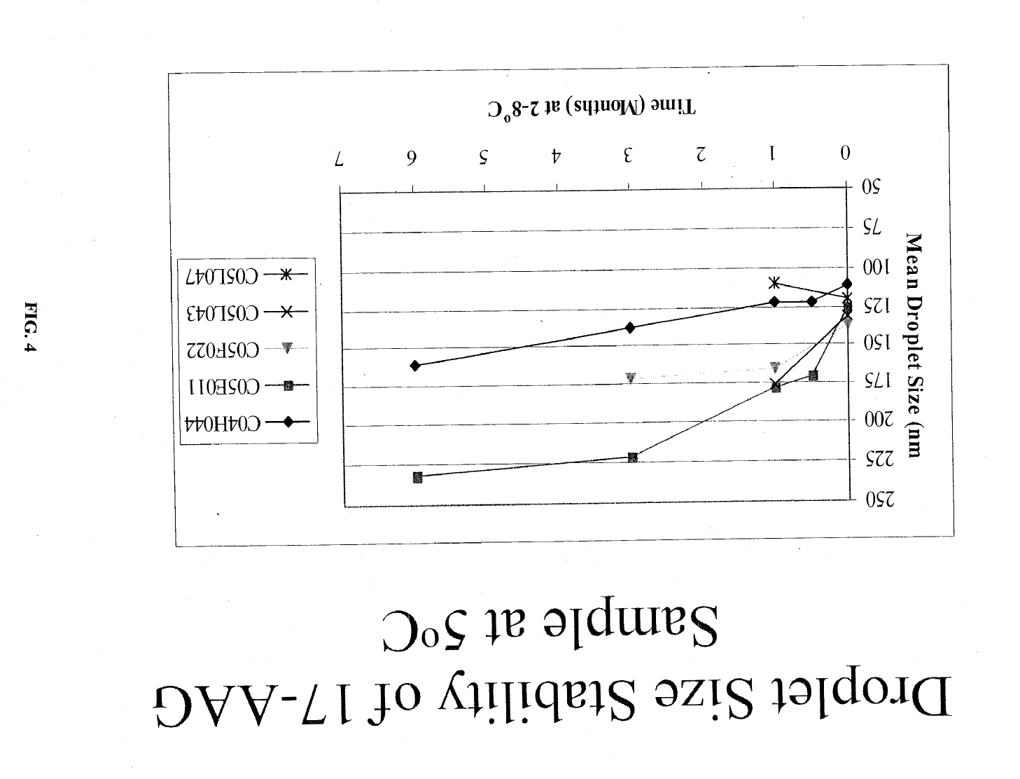

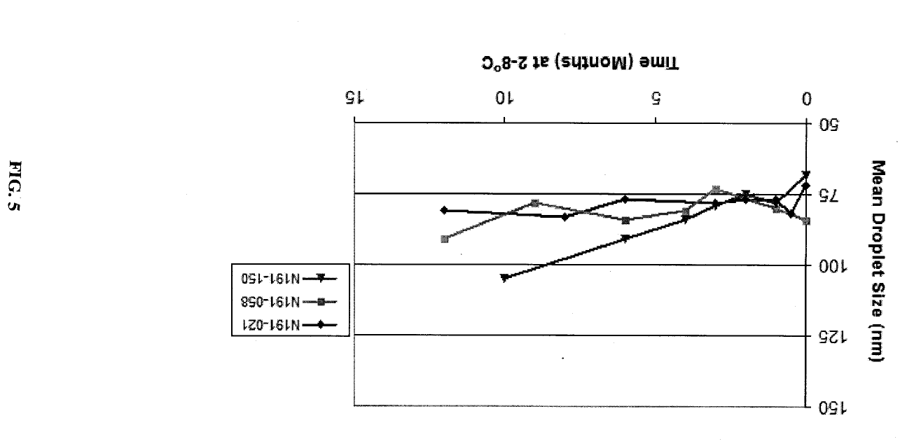

Because compositions without oleic acid have unacceptable shelf life under refrigerated storage conditions and have limited room temperature stability (less than one week), they need to be stored frozen (or lyophilized) to maintain stability periods longer than one month. In comparison, compositions with oleic acid can be stored at refrigerated temperature and room temperature for significantly longer periods of time (shelf life of 1-2 years at refrigerated state and stability maintained at room temperature for a month or more). See FIG. 3 showing the droplet size stability of compositions with and without oleic acid at room temperature. Further, compositions containing oleic acid show less variability between lots. See FIG. 4 and FIG. 5 which show effect of oleic acid on droplet size stability of compositions with and without oleic acid at refrigerated temperature.

Ansamycins may not be chemically stable in oil/water emulsions, and 17-AAG degrades in a temperature dependent manner to RS-A, an unidentified degradation product and 17-aminogeldanamycin (17-AG), which is also an active metabolite. 17-AG appears to form at a rate of about 1.7% per year, and RS-A forms at about 0.6% per year in a composition of the present invention. At these formation rates of RS-A and 17-AG, a composition of the present invention is projected to permit refrigerated storage in accordance with the current specifications (less than or equal to 2.5% and 7.5% w/w for RS-A and 17-AG, respectively) for up to two years.

The following examples are offered by way of illustration only and are not intended to be limiting of the invention.

### EXAMPLES

### Example 1

Preparation of 17-AAG; Alternative 1

To 45.0 g (80.4 mmol) of geldanamycin in 1.45 L of dry THF in a dry 2 L flask was added drop-wise over 30 minutes, 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: Rf=0.40; (5% MeOH-95%CHCl3); 17-AAG: purple: Rf=0.42 (5% MeOH-95% CHCl3)]. The solvent was removed by rotary evaporation and the crude material was slurried in 420 mL of H2O:EtOH (90:10) at 25° C., filtered and dried at 45° C. for 8 hr to give 40.9 g (66.4 mmol) of 17 purple crystals (82.6% yield, >98% pure by HPLC monitored at 254 nm). MP 206-212° C. as determined using differential scanning colorimetry (DSC). 1H NMR and HPLC are consistent with the desired product.

### Example 2

Preparation of a Low Melting Point Form of 17-AAG

An alternative method of purification is to dissolve the crude 17-AAG from example 1 in 800 mL of 2-propyl alcohol (isopropanol) at 80° C. and then cool to room temperature. Filtration followed by drying at 45° C. for 8 hr gives 44.6 g (72.36 mmol) of 17-AAG as purple crystals (90% yield, >99% pure by HPLC monitored at 254 nm). MP 147-175° C. as determined using differential scanning colorimetry (DSC). 1H NMR and HPLC are consistent with the desired product.

### Example 3

Preparation of a Low Melting Point Form of 17-AAG, Alternative 1

An alternative method of purification is to slurry the 17-AAG product from example 2 in 400 mL of H2O:EtOH (90:10) at 25° C., filtered and dried at 45° C. for 8 hr to give 42.4 g (68.6 mmol) of 17-AAG as purple crystals (95% yield, >99% pure by HPLC monitored at 254 nm). MP 147-175° C. 1H NMR and HPLC are consistent with the desired product.

### Example 4

Preparation of Other Ansamycins for Similar Formulation Ansmaycins other than 17-AAG

Essentially any ansamycin can be substituted for 17-AAG and formulated as described in the above examples. Various such ansamycins and their preparation are detailed in PCT/US03/04283. The preparation of two of these are described below.

Compound 563: 17-(benzoyl)-aminogeldanamycin. A solution of 17-aminogeldanamycin (1 mmol) in EtOAc was treated with Na₂SO₄ (0.1 M, 300 ml) at RT. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This latter was dissolved in anhydrous THF and transferred via cannula to a mixture of benzoyl chloride (1.1 mmol) and MS4A ANG. (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH2Cl2:EtOAc:MeOH. Mp=218-220° C. 1H NMR (CDCl3) 0.94 (t, 6H), 1.70 (br s, 2H), 1.79 (br s, 4H), 2.03 (s, 3H), 2.56 (dd, 1H), 2.64 (dd, 1H), 2.76-2.79 (m, 1H), 3.33 (br s, 7H), 3.44-3.46 (m, 1H), 4.325 (d, 1H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

Compound 237: A dimer. 3,3′-diamino-dipropylamine (1.32 g, 9.1 mmol) was added dropwise to a solution of Geldanamycin (10 g, 17.83 mmol) in DMSO (200 ml) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; mp 165° C.; 1H NMR (CDCl3) 0.97 (d, J=6.6 Hz, 6H, 2CH3), 1.0 (d, J=6.6 Hz, 6H, 2CH3), 1.72 (m, 4H, 2CH2), 1.78 (m, 4H, 2CH2), 1.80 (s, 6H, 2CH3), 1.85 (m, 2H, 2CH), 2.0 (s, 6H, 2CH3), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH2), 3.26 (s, 6H, 2OCH3), 3.38 (s, 6H, 20CH3), 3.40 (m, 2H, 2CH), 3.60 (m, 4H, 2CH2), 3.75 (m, 2H, 2CH), 4.60 (d, J=10 Hz, 2H, 2CH), 4.65 (Bs, 2H, 20H), 4.80 (Bs, 4H, 2NH2), 5.19 (s, 2H, 2CH), 5.83 (t, J=15 Hz, 2H, 2CH.dbd.), 5.89 (d, J=10 Hz, 2H, 2CH.dbd.), 6.58 (t, J=15 Hz, 2H, 2CH.dbd.), 6.94 (d, J=10 Hz, 2H, 2CH.dbd.), 7.17 (m, 2H, 2NH), 7.24 (s, 2H, 2CH.dbd.), 9.20 (s, 2H, 2NH); MS (m/z) 1189 (M+H).

The corresponding HCl salt was prepared by the following method: an HCl solution in EtOH (5 ml, 0.123N) was added to a solution of compound #237 (1 gm as prepared above) in THF (15 ml) and EtOH (50 ml) at room temperature. The reaction mixture was stirred for 10 min. The salt was precipitated, filtered and washed with large amount of EtOH and dried in vacuo.

### Example 5

Preparation of a 17-AAG Composition with Oleic Acid

This method can be used with any of the ansamycins prepared in Examples 1-4. The description below refers to a typical preparation of a 100 kg batch of a 17-AAG composition.

Oil Phase (Prepared in 2% Excess of Batch Requirements)

Miglyol 812N (9894 g), soybean oil (3366 Kg) and oleic acid (204 g) are mixed for about 5 minutes in a 25 L 316 L stainless steel tank using a Silverson high shear mixer. Phospholipon 90G (PL90G; 6732) is slowly added to the mixing oils. Mixing continues until the PL90G is dissolved yielding a clear viscous yellow solution. 17-AAG is weight adjusted for purity and to include a 3% excess (217.3 g) to account for degradation during manufacturing. 17-AAG is added to the oil phase and mixed using the Silverson high shear mixer until the 17-AAG has dissolved (about one hour). The 17-AAG oil phase is then filtered at 40° C. through a 5 inch capsule filter containing a 1.0/0.5 μm mixed cellulose ester filter membrane to remove any particulates that may interfere with the emulsification process. The composition of the 17-AAG oil phase is: 1.06% 17-AAG;

1.00% oleic acid; 16.49% soybean oil; 32.98% PL90G; and 48.47% Miglyol 812N.

Aqueous Phase

The aqueous phase is prepared separately from the oil phase. Water for Injection (71.5 Kg) is added to a 150 L tank. With an overhead mixer mounted in the tank, sucrose (7500 g) is added to the vortex followed by EDTA (5.0 g). The aqueous phase is mixed until all sucrose and EDTA are dissolved. The composition (% w/w) of the aqueous phase is: 9.38% sucrose; 0.0063% EDTA; and 90.62% water.

Primary Emulsion

The aqueous phase tank is connected to an in-line high shear mixer and mixing is initiated. The 17-AAG-containing oil phase is transferred via a peristaltic pump to the mixing aqueous phase to form the primary emulsion. The addition takes about 30 minutes and mixing continues for an additional 10 minutes after the 17-AAG-containing oil phase has been transferred.

While mixing with an in-line mixer, the pH of the primary emulsion is adjusted from about 5.0 to about 7.5±0.3 using 0.1N NaOH. Water for Injection is added to q.s. to 100 kg.

Microfluidization (Size Reduction)

The primary emulsion is chilled to less than 15° C., then microfluidized using a single discrete pass into another 150 L tank. Microfluidization continues until the mean droplet size of the emulsion is less than or equal to 80 nm. The product temperature is maintained at less than 15° C. during microfluidization. The microfluidized emulsion is then filtered through a 1.0/0.2 μm capsule filter containing mixed cellulose ester filter membrane.

Filtration and Filling

The emulsion is then sterile filtered through capsule prefilters (1.0/0.2 μm MCE filter membrane) and two sterilizing grade Durapore capsule filter (polyvinylidine fluoride filter membrane) arranged in series into the aseptic filling area where the product is filled (20 mL) into 20 mL Type 1 clear glass vials and then sealed with bromobutyl rubber stoppers and aluminum flip-off seals.

TABLE 2
Composition of Example 5

| Ingredient | Composition (% w/w) |
|---|---|
| 17-allyalamino-17-demethoxy-geldanamycin (17- | 0.2 |
| AAG) | |
| Miglyol 812, NF (Medium Chain Triglycerides) | 9.7 |
| Soybean Oil, USP (Long Chain Triglycerides) | 3.3 |
| Phospholipon 90G (Soy lecithin) | 6.6 |
| Oleic Acid, NF | 0.2 |
| Sucrose, NF | 7.5 |
| EDTA, USP | 0.005 |
| Sodium Hydroxide, NF | To adjust pH |
| Water for Injection, USP | q.s. |

Compositions of the present invention could also be prepared using methods described in the related applications. The following example illustrates how Ex. 4 of US 2006/0014730 and US 2006/0148776 could be modified to generate a composition of this invention.

### Example 6

Another Preparation of a 17-AAG Composition with Oleic Acid

17-AAG (or any ansamycin as described in Ex. 1-4 above) is weighed in a 5 L polypropylene beaker. Ethanol is added in an amount approximately 50× the weight of 17-AAG to phospholipid and mixed until dissolution is complete. 17-AAG is then added to the ethanol/phospholipid solution and mixed until dissolution is complete. Miglyol 812N, soy bean oil and oleic acid are then added to the solution. A sonicator bath and/or heat to approximately 45° C. may be used to help dissolve the solids. The solution may be checked using an optical microscope to ensure desired dissolution.

A stream of dry air or nitrogen gas is forced over the liquid surface in combination with vigorous stirring to evaporate the ethanol until the ethanol content is reduced to, for example, less than 50% (e.g., less than 5-10%) of its initial presence w/w. The solution can be checked under an optical microscope equipped with polarizing filters to ensure complete dissolution of 17-AAG (no crystals or precipitate).

EDTA (disodium, dihydrate, USP), sucrose, and water for injection (together, the aqueous phase) are weighed into a 5 L polypropylene beaker and stirred until the solids are dissolved. The aqueous phase is then added to the oil phase and thorough mixing effected using a high-speed emulsifier/homogenizer equipped with an emulsion head at 5000 rpm until the oil adhering to the surface is “stripped off.” Shearing rate is then increased to 10000 rpm for 2-5 minutes to generate a uniform primary emulsion. Laser light scattering (LLS) may be used to measure the average droplet size, and the solution may further be checked, e.g., under an optical microscope to determine the relative presence or absence of crystals and solids.

The emulsion pH is adjusted to 6.0±0.2 with 0.2 N NaOH. The primary emulsion is then passed through a Model 11OS microfluidizer (Microfluidics Inc., Newton, Mass., USA) operating at static pressure of about 110 psi (operating pressure of 60-95 psi) with a 75-micron emulsion interaction chamber (F20Y) for 6-8 passages until the average droplet size is less than or equal to 190 nm. LLS may be used following the individual passages to evaluate progress. The solution may further be checked for the presence of crystals using polarized light under an optical microscope.

In a laminar flow hood, the emulsion is then passed across a 0.45 micron Gelman mini capsule filter (Pall Corp., East Hills, N.Y., USA), and then across a sterile 0.2 micron Sartorius Sartobran P capsule filter (500 cm²) (Sartorius AG, Goettingen, Germany). Pressure up to 60 psi is used to maintain a smooth and continuous flow. Filtrate is then collected and a small amount could be set aside for testing using laser light scattering (LLS) and/or high performance liquid chromatography (HPLC).

### BIOLOGY EXAMPLES

### Example 7

Comparative Pharmacokinetics (17-AAG) in the Rat Following IV Administration of Formulation A (without Oleic Acid) and Formulation B (with Oleic Acid)

Summary

The pharmacokinetics (PK) of 17-(allylamino)-17-demethoxygeldanamycin (17-AAG) and its active metabolite (17-AG) were evaluated in rats after the intravenous (i.v.) administration formulations A and B. Formulation A is an oil (medium and long chain triglycerides and soy lecithin)-in-water emulsion formulation of 17-AAG. Formulation B has the same composition as formulation A, except it contains the additional ingredient of oleic acid at a final concentration of 0.2% (w/w).

Seven jugular-vein-catheterized female Sprague-Dawley rats received a single 2-minute i.v. infusion of Formulation A (n=3) or Formulation B (n=4) via the tail vein at a dose of 10 mg/kg. Each animal was bled from the catheter prior to dosing and at ten intervals after dosing. Serum concentrations of 17-AAG and 17-AG were determined using a standardized LC/MS/MS method. The individual animal 17-AAG and 17-AG concentration-versus-time curves were analyzed using non-compartmental methods.

The mean PK parameters for 17-AAG and 17-AG following administration of Formulation A and Formulation B were not significantly different.

The metabolite, 17-AG, is a product of CYP3A4 mediated oxidation of 17-AAG and thus its appearance in the plasma is dependent upon the release of 17-AAG from the emulsion droplets followed by diffusion of free 17-AAG into hepatocytes. The observations of an identical 17-AG Tmax and similar 17-AG AUC and concentration versus time profiles following administration of the two formulations suggests that the rate and extent of 17-AAG release and subsequent liver distribution are not altered by the inclusion of oleic acid in the formulation.

In summary, the data presented below indicate that the presence of oleic acid in Formulation B (according to one embodiment of the present invention) does not alter the PK of 17-AAG and its active metabolite 17-AG from that observed with Formulation A upon i.v. administration to rats.

Abbreviations

i.v. intravenous
Cₘₐₓ maximum serum concentration
Tₘₐₓ Time of Cₘₐₓ
Clₜₒₜ Total clearance
Formulation A medium and long chain triglycerides and soy lecithin)-in-water formulation of 17-AAG
Formulation B medium and long chain triglycerides, soy lecithin and oleic acid 0.2% (w/w)-in-water formulation of 17-AAG
PK pharmacokinetics
17-AAG 17-(allylamino)-17-demethoxygeldanamycin
17-AG 17-(amino)-17-demethoxygeldanamycin
AUC₍₀₋ₜₗₐₛₜ₎ Area Under the Plasma Concentration Time Curve from zero to the time of the last measurable concentration.
V_{dss} steady state volume of distribution

Formulation A is an oil (medium and long chain triglycerides and soy lecithin)-in-water emulsion formulation of 17-AAG. Formulation B has the same composition a formulation A, except it contains the additional ingredient of oleic acid at a final concentration of 0.2% (w/w). The purpose of this study was to compare the PK of 17-AAG and its active metabolite 17-AG after i.v. administration of Formulation A and Formulation B in the rat.

Materials and Methods

Formulation A was frozen at −20° C. following manufacture, thawed overnight at 4° C. on the evening prior to the in vivo study, and transferred to room temperature for about 2 hrs prior to use. Formulation B was stored at 4° C. following manufacture and transferred to room temperature for about 2 hrs prior to use. The 17-AAG concentration and emulsion droplet size were determined for each test article at the time of manufacture as described below.

Analysis of Dose Samples for 17-AAG Concentration and Droplet Size

The standardized methodology to determine the 17-AAG concentration was conducted on a HPLC system consisting of an Agilent 1100 series binary pump, Agilent 1100 series autosampler, Agilent 100 series MWV UV detector, and a Zorbax 300SB-C18, 3.5 μm particle size column (4.6 mm×150 mm). Absorbance was monitored at 332 nm. The injection volume was 50 μL and the mobile phase flow rate was 1.0 mL/min. The isocratic mobile phase was prepared by combining 480 mL 20 mM Tris-HLC (pH 7.0) with 520 mL acetonitrile. A sample of each test article was diluted 20-fold in methanol prior to HPLC analysis.

The average emulsion droplet size was measured by laser light scattering spectroscopy (LLS) using a Nanotrac 150 (Microtrac) with Microflex ver. 10.1.1 software (Microtrac). The batch sample was diluted 100-fold in de-ionized water prior to analysis.

Test System

The jugular vein catheterized female Sprague-Dawley rats used were obtained from Charles River Laboratories Inc, Portage Mich. The body weights upon dosing (Feb. 25, 2005) ranged from 268.5 to 283.6 grams with means of 270.5 and 274.9 grams for rats dosed with Formulation A and Formulation B respectively.

Experimental Design

Seven jugular-vein-catheterized female Sprague-Dawley rats received a single 2-minute i.v. infusion of Formulation A (N=3) or Formulation B (n=4) via the tail vein at a dose of 10 mg/kg (60 mg/m²). Prior to dosing, the animals were placed on a heating pad (about 40° C.) for approximately 5 minutes to promote vasodilatation. The rats were then manually restrained (Rodent Restraint Cone, Fisher Scientific) on a heating pad (about 40° C.) and the test articles were administered as a controlled 2-minute infusion (Harvard Apparatus Model 22 Infusion pump) into a tail vein using a Terumo Surflo® winged infusion set (27G×½″). The dose volumes administered (4.55 and 5.26 mL/kg of Formulation A and Formulation B, respectively) were based on the body weight determined on the day of dosing and the 17-AAG concentration of the formulations determined at the time of manufacture. Blood samples (about 250 μL) were collected from the jugular vein catheter prior to dosing, and then at 1, 5, 10, 15 and 30 minutes and at 1, 2, 3, 4 and 6 hours after dosing. The catheters were flushed with saline for injection (about 250 μL) following each blood sample. The blood was transferred to polypropylene micro-centrifuge tubes and allowed to clot for about 10 minutes at room temperature, after which they were kept on ice until centrifugation. The blood was centrifuged at 10,000×g for 10 minutes and the serum was transferred to clean micro-centrifuge tubes at stored at −20° C. until analysis.

Determination of 17-AAG and 17-AG Concentration by LC/MS/MS:

A standardized LC/MS/MS assay was used to determine the concentration of 17-AAG and 17-AG. The assay was conducted on a Thermo Finnigan LC Surveyor High Performance Liquid Chromatogram (HPLC) system (consisting of gradient pump, solvent degasser, PDA detector, column heater, and an autosampler) coupled with LCQ Deca Ion Trap mass-spectrometer. Analytes were chromatographed on Phenomenex Synergi RP-MAX C12, 4 μm particle size column (75 mm×2.0 mm). A gradient method was used with mobile phase A consisting of water (1.0% acetic acid). Mobile phase B was composed of acetonitrile (1.0% acetic acid). After equilibration with 50% A/50% B, the mobile phase mixture was changed to 2% A/98% B for 5 minutes with a total run time of 15 minutes. The flow rate was 0.4 mL/min and the column was maintained at 30° C. Absorbance of both analyte was monitored at 335 nm.

Stock solutions of 17-AAG and 17-AG were serially diluted in methanol to obtain spiking standard solutions ranging from 0.3 to 30 μg/mL. Calibration standards for 17-AAG and 17-AG were prepared by spiking solutions of 17-AAG and 17-AG dissolved in methanol into rat serum (BioChemed Pharmacologicals).

Calibration standards and samples were prepared for analysis by protein precipitation in acetonitrile followed by centrifugation and organic layer evaporation. Mobile phase reconstituted extracts were analyzed by high performance liquid chromatography coupled with mass spectrometry (HPLC/MS²-SRM) using electrospray ionization in the negative ion mode. A six point standard curve for 17-AAG (50 to 5000 ng/mL) and five point standard curve for 17-AG (50 to 3000 ng/mL) in duplicate and four quality control standards in triplicate were used for quantitation.

The lower limit of quantitation of the method was 50 ng/mL for both analytes. Individual 17-AAG and 17-AG concentration data are presented in Appendix A. Representative standard curve and chromatograms are shown in Appendix B.

Pharmacokinetic Analysis:

The individual animal 17-AAG concentration-versus-time data were analyzed using compartmental methods (WinNonlin, Version 4.1). The Terminal half-life (t_{1/2}), area under the concentration versus time curve from 0 to infinity (AUC_{0-∞}), total clearance (Cltot), and steady state volume of distribution (V_{dss}) were determined. For 17-AG, concentration-versus-time data profiles were analyzed using a non-compartmental method (WinNonlin, Version 4.1) and t_{1/2} and area under the curve from 0 to the last measurable concentration (AUCtlast) were estimated. The 17-AAG and 17-AG Cmax and Tmax values reported are the observed values. PK parameter values for Formulation A and Formulation B were compared using students t-test assuming equal variance (Microsoft Excel 2000 version 9.0.6926 SP-3).

Results

The 17-AAG concentrations of the Fonnulatuin A and Formulation B used for this study were 2.25 and 1.90 mg/mL, respectively. The mean emulsion droplet sizes were 105 nm and 60 nm for Formulation A and Formulation B, respectively.

The individual rat 17-AAG and 17-AG serum concentration data appears in Table 4.

TABLE 3
Summary of 17-AAG Pharmacokinetic Parameters

| | | FORMULA- | | |
|---|---|---|---|---|
| | | TION A | FORMULATION B | T-test |
| Parameter | Units | Mean (±SD) | Mean (±SD) | P value |
| Cₘₐₓ | ng/mL | 6243 (611) | 9361 (4866) | 0.33 |
| AUC_{(0-∞)} | ng/mL * hr | 2464 (276) | 3119 (1176) | 0.4 |
| V_{dss} | L/kg | 4.1 (0.9) | 2.9 (1.2) | 0.18 |
| Clₜₒₜ | L/hr/kg | 4.1 (0.5) | 3.5 (1.1) | 0.46 |
| t_{1/2} | Hours | 1.7 (0.1) | 1.5 (0.2) | 0.08 |

TABLE 4
Summary of 17-AG Pharmacokinetic Parameters

| | | FORMULA- | FORMULA- | |
|---|---|---|---|---|
| | | TION A | TION B | T-test |
| Parameter | Units | Mean (±SD) | Mean (±SD) | P value |
| Cₘₐₓ | ng/mL | 230 (13) | 236 (81) | 0.9 |
| Tₘₐₓ^{a} | hr | 0.05 (0.0) | 0.05 (0.0) | NA |
| AUC₍₀₋ₜₗₐₛₜ₎ | ng/mL * hr | 273 (4) | 343 (71) | 0.16 |
| 17-AG AUC as | % | 11.2 (1.4) | 11.8 (3.7) | 0.80 |
| percent of | | | | |
| 17-AAG AUC | | | | |
| t_{1/2} | Hours | 4.0 (0.4) | 3.6 (0.3) | 0.13 |

^{a}Measured from initiation of infusion
NA = not applicable

The mean PK parameter estimates for 17-AAG (Table 3) and 17-AG (Table 4) were not significantly different following administration of Formulation A and Formulation B. The individual rat PK parameters are presented in Tables 5-7. Following administration of both formulations, the Tₘₐₓ of the active metabolite 17-AG occurred at 1 minute post infusion and the ratios of the metabolite to parent AUC's were not significantly different.

The metabolite 17-AG is a product of CYP3A4 mediated oxidation of 17-AAG (Conforma Therapeutics Technical Report 00-1010-PC/PK-TR-006-A) and thus its appearance in the plasma is dependent upon the release of 17-AAG from the emulsion droplets followed by diffusion of free 17-AAG into hepatocytes. The observations of an identical 17-AG Tₘₐₓ and similar 17-AG AUC and concentration versus time profiles following administration of the two formulations suggests that the rate and extent of 17-AAG release and subsequent liver distribution are not altered by the inclusion of oleic acid in the formulation.

In summary, these data indicate that the presence of oleic acid in Formulation B does not alter the PK of 17-AAG and its active metabolite 17-AG from that observed with FORMULATION A upon i.v. administration to rats.

TABLE 5
17-AAG Serum Concentration Data (ng/mL in Serum)

| Sample | FORMULATION B | | | | FORMULATION A | | |
|---|---|---|---|---|---|---|---|
| Time | Rat 1 | Rat 2 | Rat 3 | Rat 4 | Rat 5 | Rat 6 | Rat 7 |
| Pre Dose | ND | ND | ND | ND | ND | ND | ND |
| 1 min | 16402 | 8620 | 6939 | 5483 | 6678 | 5544 | 6506 |
| 5 min | 7088 | 6049 | 3217 | 691 | 4649 | 3466 | 4219 |
| 10 min | 5746 | 4056 | 3024 | 2356 | 3338 | 2096 | 3275 |
| 15 min | 4254 | 3451 | 987 | 1709 | 2029 | 1775 | 2317 |
| 30 min | 1974 | 1334 | 1193 | 1173 | 1081 | 995 | 1333 |
| 1 hr | 1065 | 533 | 510 | 521 | 395 | 411 | 629 |
| 2 hrs | 295 | 211 | 160 | 122 | 115 | 135 | 121 |
| 3 hrs | 83 | 128 | 98 | 65 | 176 | 111 | 122 |
| 4 hrs | 44 | 50 | 43 | 41 | 52 | 55 | 40 |
| 6 hrs | 22 | 22 | 21 | ND | 30 | 28 | 22 |

ND = Not Detected

TABLE 6
17-AG Serum Concentration Data (ng/mL in Serum)

| Sample | FORMULATION B | | | | FORMULATION A | | |
|---|---|---|---|---|---|---|---|
| Time | Rat 1 | Rat 2 | Rat 3 | Rat 4 | Rat 5 | Rat 6 | Rat 7 |
| Pre Dose | ND | ND | ND | ND | ND | ND | ND |
| 1 min | 356 | 209 | 196 | 183 | 245 | 221 | 223 |
| 5 min | 191 | 173 | 149 | 129 | 167 | 149 | 173 |
| 10 min | 228 | 165 | 152 | 159 | 165 | 141 | 160 |
| 15 min | 245 | 175 | 142 | 157 | 142 | 135 | 144 |
| 30 min | 137 | 100 | 77 | 150 | 59 | 83 | 69 |
| 1 hr | 164 | 46 | 68 | 124 | 46 | 56 | 52 |
| 2 hrs | 53 | 42 | 49 | 45 | 41 | 41 | 43 |
| 3 hrs | 45 | 47 | 47 | 49 | 50 | 44 | 47 |
| 4 hrs | 27 | 21 | 23 | 29 | 23 | 25 | 22 |
| 6 hrs | 25 | 21 | 21 | 23 | 23 | 23 | 21 |

ND = Not Detected

TABLE 7

| | | 17-AAG PK Parameters | | | | | 17-AG PK Parameters | | | |
|---|---|---|---|---|---|---|---|---|---|---|
| | | AUC | Cₘₐₓ | T_{1/2} | V_{dss} | Clₜₒₜ | AUC | Cₘₐₓ | T_{1/2} | Tₘₐₓ |
| Rat | Formulation | (ng/mL × hr) | (ng/mL) | (hr) | (L/kg) | (L/hr/kg) | (ng/mL × hr) | (ng/mL) | (hr) | (hr) |
| 1 | FORMULATION B | 4737 | 16402 | 1.7 | 1.5 | 2.1 | 432 | 356 | 3.8 | 0.05 |
| 2 | | 3242 | 8620 | 1.3 | 2.4 | 3.1 | 281 | 209 | 3.7 | 0.05 |
| 3 | | 2280 | 6939 | 1.5 | 4.0 | 4.4 | 290 | 196 | 3.2 | 0.05 |
| 4 | | 2217 | 5483 | 1.3 | 3.6 | 4.5 | 369 | 183 | 3.7 | 0.05 |
| 5 | FORMULATION A | 2532 | 6678 | 1.9 | 4.1 | 3.9 | 269 | 245 | 4.2 | 0.05 |
| 6 | | 2160 | 5544 | 1.7 | 5.0 | 4.6 | 277 | 221 | 4.3 | 0.05 |
| 7 | | 2698 | 6506 | 1.6 | 3.2 | 3.7 | 272 | 223 | 3.6 | 0.05 |
| Mean | FORMULATION B | 3119 | 9361 | 1.5 | 2.9 | 3.5 | 343 | 236 | 3.6 | 0.05 |
| STD | | 1176 | 4886 | 0.2 | 1.2 | 1.1 | 71 | 81 | 0.3 | 0.00 |
| Mean | FORMULATION A | 2464 | 6242 | 1.7 | 4.1 | 4.1 | 272 | 230 | 4.0 | 0.05 |
| STD | | 276 | 611 | 0.1 | 0.9 | 0.5 | 4 | 13 | 0.4 | 0.00 |

All documents cited herein are indicative of the levels of skill in the art to which the invention pertains and are incorporated by reference herein in their entireties. None, however, is admitted to be prior art. Other embodiments are within the following claims.

What is claimed is:

1. A pharmaceutical composition comprising an oil phase and an aqueous phase, the oil phase comprising an ansamycin and less than 2% w/w oleic acid, wherein the ansamycin is geldanamycin, 17-aminogeldanamycin, 17-allyalamino-17-demethoxy-geldanamycin, compound 563, or compound 237 having the structures below, or a salt of any one of the aforementioned ansamycins.

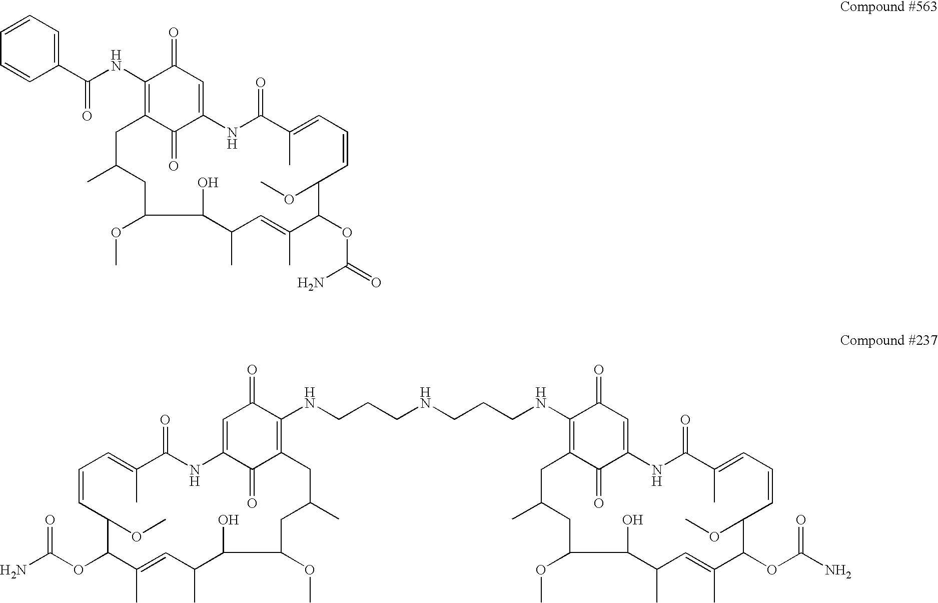

2. The pharmaceutical composition of claim 1, wherein the ansamycin is 17-allyalamino-17-demethoxy-geldanamycin.

3. The pharmaceutical composition of claim 1, wherein the final concentration of the ansamycin ranges between about 0.5 to 4 mg/mL.

4. The pharmaceutical composition of claim 1, wherein the final concentration of the ansamycin ranges between about 1 to 3 mg/mL.

5. The pharmaceutical composition of claim 1, wherein the final concentration of the ansamycin is about 2 mg/mL.

6. The pharmaceutical composition of claim 1, wherein the amount of oleic acid in the composition is no more than about 1% w/w of the pharmaceutical composition.

7. The pharmaceutical composition of claim 1, wherein the amount of oleic acid in the composition is between about 0.5% to 0.05% w/w of the pharmaceutical composition.

8. The pharmaceutical composition of claim 1, wherein the amount of oleic acid in the composition is about 0.2% w/w of the pharmaceutical composition.

9. The pharmaceutical composition of claim 1, further comprises medium chain triglycerides.

10. The pharmaceutical composition of claim 9, wherein the amount of the medium chain triglycerides is no more than about 15% w/w of the pharmaceutical composition.

11. The pharmaceutical composition of claim 9, wherein the amount of the medium chain triglycerides ranges between about 7% to 13% w/w of the pharmaceutical composition

12. The pharmaceutical composition of claim 9, further comprises long chain triglycerides.

13. The pharmaceutical composition of claim 12, wherein the amount of the long chain triglycerides is no more than about 7% w/w of the pharmaceutical composition.

14. The pharmaceutical composition of claim 12, wherein the amount of the long chain triglycerides ranges between about 2% to 5% w/w of the pharmaceutical composition.

15. The pharmaceutical composition of claim 1, further comprises an emulsifying agent.

16. The pharmaceutical composition of claim 15, wherein the emulsifying agent is lecithin.

17. The pharmaceutical composition of claim 16, wherein the emulsifying agent is soy lecithin.

18. The pharmaceutical composition of claim 15, wherein the amount of lecithin ranges between about 3% to 10% w/w of the pharmaceutical composition.

19. The pharmaceutical composition of claim 15, wherein the amount of lecithin ranges between about 5% to 8% w/w of the pharmaceutical composition.

20. The pharmaceutical composition of claim 1, wherein the oil phase is about 5% to 30% w/w of the pharmaceutical composition.

21. The pharmaceutical composition of claim 2, wherein the amount of oleic acid in the composition is between about 0.5% to 0.05% w/w.

22. The pharmaceutical composition of claim 5, wherein the ansamycin is 17-allyalamino-17-demethoxy-geldanamycin and wherein the amount of oleic acid in the composition is about 0.2% w/w of the pharmaceutical composition.

23. The pharmaceutical composition of claim 1, wherein the final concentration of the ansamycin ranges between about 1 to 3 mg/mL; the amount of oleic acid in the composition is between about 0.5% to 0.05% w/w; the amount of the medium chain triglycerides ranges between about 7% to 13% w/w; the amount of the long chain triglycerides ranges between about 2% to 5% w/w; and the amount of lecithin ranges between about 5% to 8% w/w of the pharmaceutical composition.

24. The pharmaceutical composition of claim 1, wherein the final concentration of the ansamycin is about 2 mg/mL; the amount of oleic acid in the composition is about 0.2% w/w; the amount of the medium chain triglycerides ranges between about 7% to 13% w/w; the amount of the long chain triglycerides ranges between about 2% to 5% w/w; and the amount of lecithin ranges between about 5% to 8% w/w, and wherein the ansamycin is 17-allyalamino-17-demethoxy-geldanamycin and the lecithin is soy lecithin.

25. The pharmaceutical composition of claim 1, wherein the mean droplet size is less than about 500 nm.

26. The pharmaceutical composition of claim 1, wherein the mean droplet size is less than about 150 nm.

27. The pharmaceutical composition of claim 1, wherein the mean droplet size is about 80 nm.

28. The pharmaceutical composition of claim 23, wherein the mean droplet size is about 80 nm.

29. The pharmaceutical composition of claim 24, wherein the mean droplet size is about 80 nm.

30. The pharmaceutical composition of claim 23, wherein the pH of the pharmaceutical composition ranges from about 5 to 8.

31. The pharmaceutical composition of claim 24, wherein the pH of the pharmaceutical composition ranges from about 5 to 8.

32. A pharmaceutical composition comprising an oil phase and an aqueous phase, the oil phase further comprising 17-allyalamino-17-demethoxy-geldanamycin and less than 2% w/w oleic acid, the pharmaceutical composition being stable at pH ranges from about 5 to 8 and temperature ranges between about 0° C. to 10° C. for at least 18 months.

33. The composition of claim 31, wherein said pH ranges between about 5.5 to 7.5 and temperature ranges between about 2° C. to 8° C.

34. The composition of claim 31, wherein the mean droplet size of said composition increases no more than 100 nm at room temperature and pH ranges from about 5 to 8 for at least 3 months.

35. The composition of claim 31, wherein the mean droplet size of said composition increases no more than 50 nm at room temperature and pH ranges from about 5.5 to 7 for at least 3 months.

36. The composition of claim 31, wherein the mean droplet size of said composition increases no more than 50 nm at temperature ranges from about 0° C. to 10° C. and pH ranges from about 5 to 8 for at least 12 months.

37. The composition of claim 31, wherein the mean droplet size of said composition increases no more than 35 nm at temperature ranges from about 2° C. to 8° C. and pH ranges from about 5.5 to 7 for at least 12 months.

38. A method of treating an individual having an HSP90 mediated disorder comprising administering to said individual an effective amount of a pharmaceutical composition of claim 1.

39. A method of treating an individual having an HSP90 mediated disorder comprising administering to said individual an effective amount of a pharmaceutical composition of claim 23.

40. A method of treating an individual having an HSP90 mediated disorder comprising administering to said individual an effective amount of a pharmaceutical composition of claim 24.

41. The method of claim 38, wherein the HSP90 mediated disorder is selected from the group consisting of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant diseases.

42. The method of claim 39, wherein the HSP90 mediated disorder is selected from the group consisting of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant diseases.

43. The method of claim 40, wherein the HSP90 mediated disorder is selected from the group consisting of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant diseases.

44. The method of claim 43, wherein the HSP90 mediated disorder is selected from the group consisting of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant diseases.

45. The method of claim 38, further comprising administering at least one therapeutic agent selected from the group consisting of cytotoxic agents, anti-angiogenesis agents and anti-neoplastic agents.

46. The method of claim 39, further comprising administering at least one therapeutic agent selected from the group consisting of cytotoxic agents, anti-angiogenesis agents and anti-neoplastic agents.

47. The method of claim 40, further comprising administering at least one therapeutic agent selected from the group consisting of cytotoxic agents, anti-angiogenesis agents and anti-neoplastic agents

48. The method of claim 47, wherein the at least one anti-neoplastic agent is selected from the group consisting of alkylating agents, anti-metabolites, epidophyllotoxins, antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors.

49. The use of a composition according to claims 1-31 in the manufacture of a medicament.

50. The use of a composition according to claims 1-31 in the manufacture of a medicament for the therapeutic and prophylactic treatment of HSP90-mediated diseases and conditions.
